(19) United States
(12) Patent Application Publication
(10) Pub. No.: US 2025/0152575 A1
(43) Pub. Date: May 15, 2025

(54) METHODS FOR TREATING IMMUNODEFICIENCY DISEASE

(71) Applicant: X4 Pharmaceuticals, Inc., Boston, MA (US)

(72) Inventors: Robert D. Arbeit, West Newton, MA (US); Paula Marie Ragan, Belmont, MA (US)

(21) Appl. No.: 18/895,123

(22) Filed: Sep. 24, 2024

Related U.S. Application Data
(63) Continuation of application No. 17/644,295, filed on Dec. 14, 2021, now abandoned.
Application No. 17/644,295 is a continuation of application No. 16/804,863, filed on Feb. 28, 2020, now Pat. No. 11,219,621.
Application No. 16/804,863 is a continuation of application No. 16/065,470, filed on Jun. 22, 2018, now Pat. No. 10,610,527.
(60) Provisional application No. 62/428,964, filed on Dec. 1, 2016.
(60) Provisional application No. 62/271,087, filed on Dec. 22, 2015.

(51) Int. Cl. A61K 31/4709; A61K 9/00; A61K 9/48; A61K 31/395; A61P 17/12; A61P 37/04; C07D 235/14; C07D 401/04; C07D 401/12

(52) U.S. Cl. CPC A61K 31/4709; A61K 9/0053; A61K 9/485; A61K 9/4858; A61K 9/4866; A61K 31/395; A61P 17/12; A61P 37/04; C07D 235/14; C07D 401/04; C07D 401/12

(57) ABSTRACT

The present invention relates to methods of treating patients with WHIM syndrome or related disorders, such as myelokathexis, in which X4P-001 is administered in order to reduce the activity of CXCR4. The methods demonstrate surprising effectiveness, with comparatively little toxicity.

### FIELD OF THE INVENTION

The present invention relates to methods for treating immunodeficiency disease, in particular, methods for treating warts, hypogammaglobulinemia, immunodeficiency, myelokathexis (WHIM) syndrome, or “WHIMS.” WHIMS is a disease characterized by neutropenia and lymphopenia resulting in skin and genital warts and recurring infections.

### CROSS-REFERENCE TO RELATED APPLICATIONS

This application is a continuation of U.S. patent application Ser. No. 16/804,863, filed Feb. 28, 2020, which is a continuation of U.S. patent application Ser. No. 16/065,470, filed Jun. 22, 2018, now U.S. Pat. No. 10,610,527, which is a 35 U.S.C. § 371 filing of International Application No. PCT/US2016/068394, filed Dec. 22, 2016, which claims the benefit of priority to United States Provisional Patent Application Ser. Nos. U.S. Ser. No. 62/271,087, filed Dec. 22, 2015, and U.S. Ser. No. 62/428,964, filed Dec. 1, 2016, the entirety of each of which is hereby incorporated by reference.

### BACKGROUND OF THE INVENTION

WHIM syndrome is a rare autosomal dominant immunodeficiency disorder which results in multiple mutations that remove 10-19 amino acids from the carboxy-terminus of CXCR4, a chemokine receptor expressed by both hematopoietic and non-hematopoietic cells [Hernandez 2003]. The mutation in the CXCR4 receptor is known to prevent the normal release of mature neutrophils from the bone marrow to the blood [Kawai 2005] resulting in neutropenia in patients with WHIM syndrome [Dale 2011]. In addition to neutropenia, WHIM syndrome is characterized by lymphopenia that affects the levels of circulating T and B cells. [Balabanian 2012, Dotta 2011] resulting in low levels of immunoglobulins. The exact mechanism for lymphopenia is not known but may be attributable to interruption of the normal trafficking of lymphocytes and their retention in the marrow and other lymphoid tissues [Ma 1999].

Generally, clinical symptoms first appear in early childhood with recurrent bacterial infections due to low levels of white blood cells and antibodies [NORD 2015]. Common infections include otitis media, cellulitis, impetigo, abscess, bacterial pneumonia, sinusitis, and periodontitis. Affected individuals are particularly susceptible to human papillomavirus (HPV), which can cause widespread warts affecting the hands, feet, face, and trunk and are often recalcitrant [NORD 2015]. Mucosal and genital warts may also develop and these warts are associated with an increased risk of progressing to cervical carcinoma [NORD 2015]. Current treatments include G-CSF and intravenous immunoglobulin but these are non-specific, expensive, difficult to administer, and only partially effective [Kawai 2009].

Present treatments available for patients with WHIM syndrome are insufficient. There is a clear unmet need for agents that improve outcomes in the treatment of such patients.

### BRIEF DESCRIPTION OF THE FIGURES

FIG. 1 illustrates X4P-001 inhibition on SDF-1α binding to CXCR4+CEM-CCRF cells.

FIG. 2 illustrates X4P-001 inhibition of SDF-1α stimulated Eu-GTP binding.

FIG. 3 illustrates X4P-001 inhibition of SDF-1α stimulated [³⁵S]-GTP-γ-S binding.

FIG. 4 illustrates X4P-001 inhibition of SDF-1α induced calcium flux.

FIG. 5 illustrates X4P-001 inhibition of SDF-1α stimulated CCRF-CEM chemotaxis.

FIG. 6 illustrates SDF-1α stimulation of calcium flux in wild-type and CXCR4 variants.

FIG. 7 illustrates X4P-001 inhibition of SDF-1α stimulation in wild-type and CXCR4 variants.

FIG. 8 illustrates white blood cell (A), neutrophil (B), and lymphocyte (C) counts following oral administration of X4P-001 to male beagle dogs.

FIG. 9 illustrates dose-dependent increases (2-3X) in WBC counts in human subjects who were administered X4P-001.

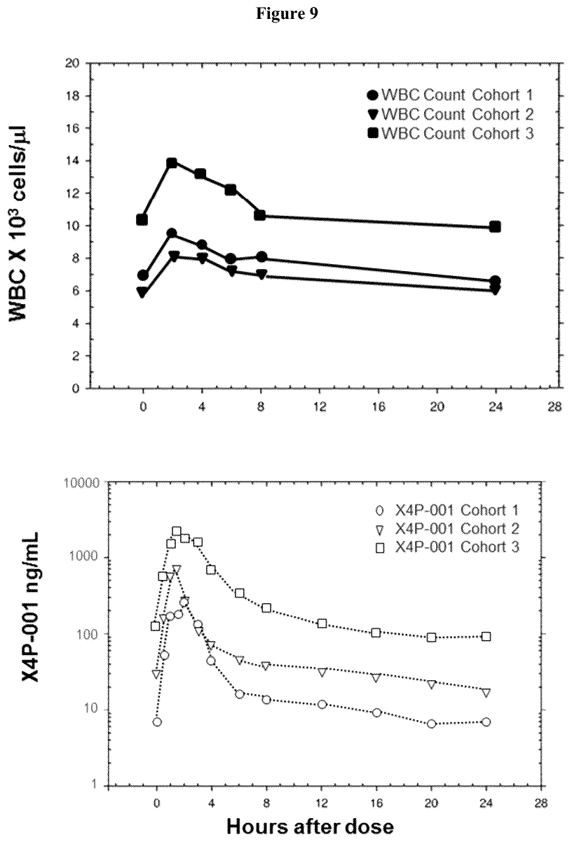

### DETAILED DESCRIPTION OF CERTAIN EMBODIMENTS OF THE INVENTION

Effective targeted treatments for WHIM syndrome, like X4P-001, are needed for the management of patients. X4P-001 can be administered orally, which in addition to being a targeted treatment, makes it an excellent candidate in a chronic treatment setting that would be required for patients with WHIM syndrome.

The ligand for the CXCR4 receptor is SDF-1α which is involved with numerous physiologic processes and plays a central role in hematopoieic cells homing to and being released from the bone marrow [Lapidot 2002]. Mutations in the CXCR4 prevent the normal release of mature neutrophils from the bone marrow into the blood [Kawai 2005]. Bone marrow examinations of patients affected with WHIM syndrome show abundant neutrophils with hyper-segmented nuclei and remnants of neutrophils in bone marrow macrophages. [Bohinjec 1981].

The disruption of the CXCR4/SDF-1α axis results in WHIM syndrome patients having low white blood cell counts usually <1.0×10⁹/L with severe neutropenia and lymphopenia present [Dale 2011]. The mechanism of CXCR4/SDF-1α axis disruption is described in the following paragraph.

CXCR4 is a G protein-coupled receptor and engagement by SDF-1α induces typical activation of G protein-dependent pathways of a chemokine receptor [Baggiolini 1998, Zlotnik 2000]. These processes are regulated in a timely manner by the recruitment of β-arrestin to the receptor that precludes further G-protein activation (ie, desensitization) and leads to receptor internalization. Mutants of CXCR4 associated with WHIM syndrome give rise to impaired desensitization and internalization of the receptor upon SDF-1α exposure, leading to enhanced and prolonged receptor activation. [Hernandez 2003, Balabanian 2005, Gulino 2004, Kawai 2005, Lagane 2008, McCormick 2009]. Because CXCR4 normally regulates leukocyte trafficking and in particular is important for neutrophil adhesion in the bone marrow, prolonging the activity of SDF-1α dependent signaling is the probable cause for myelokathexis (MKX) and neutropenia seen in WHIM syndrome. [McDermott 2011-a].

X4P-001 is a small molecule antagonist of CXCR4 having the potential to block the enhanced signaling activity of mutant CXCR4 resulting in an increase in the number of circulating white blood cells by overcoming the impaired down regulation (receptor internalization) and receptor dysfunction caused by mutant CXCR4 (McDermott 2011-b). It has also been demonstrated that X4P-001 inhibits the most common genotypic forms of CXCR4 attributable to WHIM syndrome (R334X and E343X) to a similar extent as the wild type CXCR4 [Mosi 2012].

These studies demonstrated that oral administration of up to 400 mg BID for 3.5 days (healthy volunteers) and 200 mg BID for 8-10 days (healthy volunteers and HIV patients) was well-tolerated with no pattern of adverse events or clinically significant laboratory changes. These studies also demonstrated pharmacodynamic activity, with dose- and concentration-related changes in circulating white blood cells (WBCs); and a high volume of distribution (VL), suggesting high tissue penetrance.

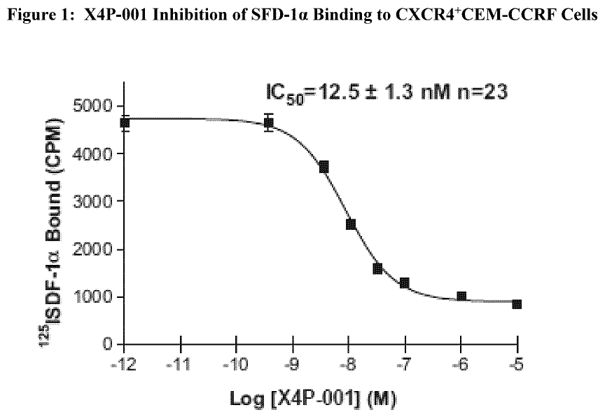

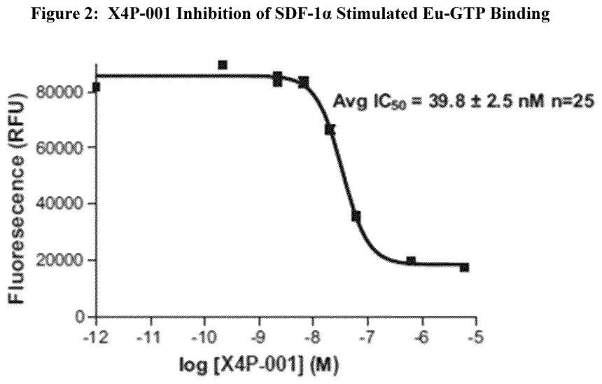

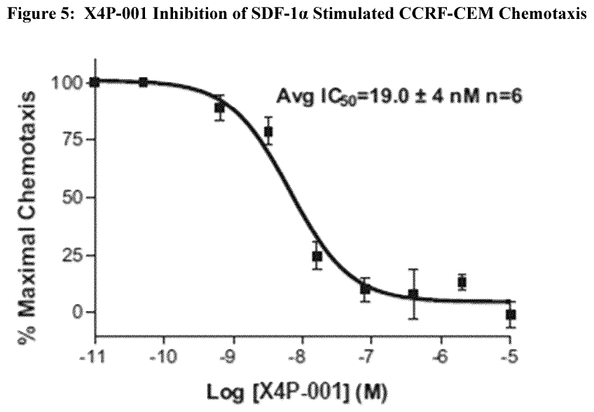

The inventors conceived that CXCR4 antagonism by X4P-001 may provide significant treatment benefits in patients with WHIMS, and individual aspects of WHIMS, which is an acronym for warts, hypogammaglobulinemia (low immunoglobulin levels), immunodeficiency (susceptibility to infections) and myelokathexis (trapping of white blood cells in the bone marrow). Administration of X4P-001 inhibits SDF-1α binding to CXCR4 and CXCR4+CEM-CCRF cells. [See FIG. 1]. Administration of X4P-001 also inhibits CXCR4 cell signaling and SDF-1α induced calcium flux. [See FIGS. 2-4]. In this manner, X4P-001 inhibits SDF-1α stimulated CCRF-CEM chemotaxis. [See FIG. 5].

Moreover, the inventors conceived that such a result might be achieved with comparatively little toxicity since CXCR4-targeted drugs are specifically targeted and do not induce cell cycle arrest in normal proliferating cell populations. Accordingly, the present invention provides significant advantages in treatment outcomes utilizing the low toxicity and effects of the CXCR4 inhibitor AMD11070 (X4P-001).

In the present invention, patients with WHIMS, or related syndromes, are treated with X4P-001, or a pharmaceutically acceptable salt or composition thereof either as a single agent (monotherapy), or in combination with another agent, such as granulocyte colony-stimulating factor (G-CSF), granulocyte-macrophage colony-stimulating factor (GM-CSF) or intravenous immunoglobulin (IVIG).

In some embodiments, the present invention provides a method for treating WHIMS in a patient in need thereof, wherein said method comprises administering to said patient X4P-001 or a pharmaceutically acceptable salt or composition thereof in combination with G-CSF, GM-CSF and/or IVIG. Other treatments that may be utilized in the treatment of WHIMS include bone marrow transplantation and treatment with cord blood stem cells.

In some embodiments, a provided method comprises administering the X4P-001, or a pharmaceutically acceptable salt or composition thereof, to a patient in a fasted state.

In certain embodiments, the present invention provides a method for treating WHIMS in a patient in need thereof, wherein said method comprises administering to said patient X4P-001, or a pharmaceutically acceptable salt or composition thereof, further comprising the step of obtaining a biological sample from the patient and measuring the amount of a disease-related biomarker. In some embodiments, the biological sample is a blood sample. In certain embodiments, the disease-related biomarker is selected from the group consisting of CXCR4, SDF-1α/CXCL12; and GRK3 (G protein coupled receptor kinase 3).

In certain embodiments, the present invention provides a method for treating WHIMS in a patient in need thereof, wherein said method comprises administering to said patient X4P-001 or a pharmaceutically acceptable salt or composition thereof.

In some embodiments, the X4P-001 or a pharmaceutically acceptable salt thereof is administered in a dose of from about 25 mg/day to about 150 mg/day.

In some embodiments, said patient exhibits warts.

In some embodiments, cells taken from the patient exhibit expression of a mutant form of CXCR4.

In some embodiments, cells taken from the patient exhibit increased expression of CXCR4.

In some embodiments, the method further comprises the step of obtaining a biological sample from the patient and measuring the amount of a disease-related biomarker.

In some embodiments, the biological sample is a blood sample.

In some embodiments, the disease-related biomarker is circulating CXCR4.

In some embodiments, the X4P-001 or a pharmaceutically acceptable salt or composition thereof is administered orally once per day.

In some embodiments, the X4P-001 or a pharmaceutically acceptable salt or composition thereof is administered orally twice per day.

In some embodiments, the present invention provides a unit dosage form comprising a composition comprising:
(a) X4P-001, or a pharmaceutically acceptable salt thereof—about 10-20% by weight of the composition;
(b) microcrystalline cellulose—about 70-85% by weight of the composition;
(d) croscarmellose sodium—about 5-10% by weight of the composition;
(e) sodium stearyl fumarate—about 0.5-2% by weight of the composition; and
(f) colloidal silicon dioxide—about 0.1-1.0% by weight of the composition.

In some embodiments, the unit dosage form is in capsule form.

In some embodiments, the capsule comprises about 25 mg X4P-001, or a pharmaceutically acceptable salt thereof.

In some embodiments, the present invention provides a method for treating WHIM syndrome in a patient in need thereof, comprising the step of administering to the patient a disclosed unit dosage form.

In some embodiments, the present invention provides a method for treating WHIM syndrome in a patient in need thereof, comprising administering to said patient X4P-001 or a pharmaceutically acceptable salt or composition thereof, in an amount effective to increase absolute neutrophil count (ANC) and/or to increase absolute lymphocyte count (ALC) in the patient, for example in the patient's blood. In some embodiments, the ANC and/or ALC is increased in the patient to about 60%, 70%, 80%, 90%, 95%, or 100% of that of an average, healthy human who does not have WHIM syndrome or another immunodeficiency. In some embodiments, the ANC and/or ALC is increased in the patient to about 60%, 70%, 80%, 90%, 95%, or 100% of that of an average, healthy human of similar age, weight, and sex to that of the patient.

In some embodiments, the present invention provides a method for treating WHIM syndrome in a patient in need thereof, comprising administering to said patient X4P-001 or a pharmaceutically acceptable salt or composition thereof, in an amount effective to increase absolute neutrophil count (ANC) to a level greater than or equal to 600/μL and/or to increase absolute lymphocyte count (ALC) to a level greater than or equal to 1000/μL.

In some embodiments, said patient originally exhibited ANC less than 600/μL and/or ALC less than 1000/μL before treatment with X4P-001.

In some embodiments, said patient originally exhibited ANC less than 400/μL and/or ALC less than 650/μL before treatment with X4P-001.

In some embodiments, a disclosed method results in increases in ANC levels to at least about 600/μL, about 800/μL, about 1000/μL, about 1,200/μL, or to about that of a human with a normally-functioning immune system, on at least 85% of assessments.

In some embodiments, a disclosed method results in increases in ALC to at least about 1000/μL, about 1,200/μL, or about 1,500/μL, or to about that of a human with a normally-functioning immune system, on at least 85% of assessments.

In some embodiments, a disclosed method results in improved levels of protective antibody in the patient in response to a vaccine.

In some embodiments, a disclosed method results in a lowered frequency of infections in the patient, such as at least 50% less infections, such as respiratory tract infections.

In some embodiments, a disclosed method results in increased levels of total circulating WBC, neutrophils, and/or lymphocytes. In some embodiments, cell counts of WBC, neutrophils, and/or lymphocytes increase to at least 1.4× baseline. In some embodiments, cell counts of WBC, neutrophils, and/or lymphocytes increase to at least 1.8× baseline. In some embodiments, cell counts of WBC, neutrophils, and/or lymphocytes increase to at least 2.9× baseline. In some embodiments, cell counts of lymphocytes increase to at least 2.9× baseline. In some embodiments, cell counts of neutrophils increase to at least 2.7× baseline and lymphocytes to 1.9× baseline.

In some embodiments, the present invention provides a method of treating WHIMS in a patient in need thereof, wherein said method comprises administering to said patient an effective amount of X4P-001 or a pharmaceutically acceptable salt or composition thereof in conjunction with another treatment for warts, HPV infection, or neutropenia.

### Dosage and Formulations

X4P-001 is a CXCR4 antagonist, with molecular formula C21H27N5; molecular Weight 349.48 amu; appearance white to pale yellow solid; solubility: X4P-001 is freely soluble in the pH range 3.0 to 8.0 (>100 mg/mL), sparingly soluble at pH 9.0 (10.7 mg/mL) and slightly soluble at pH 10.0 (2.0 mg/mL). X4P-001 is only slightly soluble in water; and melting point of 108.9° ΔC.

The chemical structure of X4P-001 is depicted below.

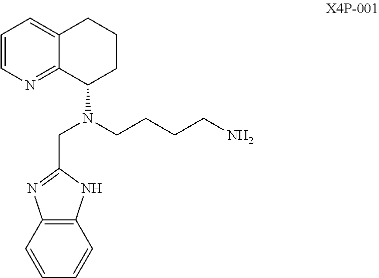 [SMILES: NCCCCN(Cc1nc2ccccc2[nH]1)[C@H]1CCCc2cccnc21]

In certain embodiments, the composition containing X4P-001 is administered orally, in an amount from about 10 mg to about 600 mg daily. In certain embodiments, the dosage composition may be provided twice a day in divided dosage, approximately 12 hours apart. In other embodiments, the dosage composition may be provided once daily. The terminal half-life of X4P-001 has been generally determined to be between about 12 to about 24 hours, or approximately 14.5 hrs. Dosage for oral administration may be from about 10 mg to about 300 mg once or twice per day. In certain embodiments, the dosage of X4P-0001 useful in the invention is from about 20 mg to about 600 mg daily. In other embodiments, the dosage of X4P-001 useful in the invention may range from about 25 mg to about 200 mg daily, from about 25 mg to about 150 mg daily, from about 25 mg to about 100 mg daily, from about 25 mg to about 50 mg daily, from about 50 mg to about 150 mg daily, or from about 50 mg to about 100 mg daily.

In some embodiments, a provided method comprises administering to the patient a pharmaceutically acceptable composition comprising X4P-001 wherein the composition is formulated for oral administration. In certain embodiments, the composition is formulated for oral administration in the form of a tablet or a capsule. In some embodiments, the composition comprising X4P-001 is formulated for oral administration in the form of a capsule.

In certain embodiments, a provided method comprises administering to the patient one or more capsules comprising 10 mg to 1200 mg X4P-001 active ingredient; and one or more pharmaceutically acceptable excipients. In certain embodiments, the capsule is comprised of hard gelatin.

In certain embodiments, the present invention provides a composition comprising X4P-001, or a pharmaceutically acceptable salt thereof, one or more diluents, a disintegrant, a lubricant, a flow aid, and a wetting agent. In some embodiments, the present invention provides a composition comprising 10 mg to 1200 mg X4P-001, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In some embodiments, the present invention provides a unit dosage form wherein said unit dosage form comprises a composition comprising 10-200 mg X4P-001, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In certain embodiments, the present invention provides a unit dosage form comprising a composition comprising X4P-001, or a pharmaceutically acceptable salt thereof, present in an amount of about 10 mg, about 20 mg, about 25 mg, about 50 mg, about 75 mg, about 100 mg, about 150 mg, about 200 mg, about 250 mg, about 300 mg, about 400 mg, about 450 mg, about 500 mg, about 600 mg, about 700 mg, about 750 mg, about 800 mg, about 900 mg, about 1000 mg, about 1100 mg, or about 1200 mg. In some embodiments, a provided composition (or unit dosage form) is administered to the patient once per day, twice per day, three times per day, or four times per day. In some embodiments, a provided composition (or unit dosage form) is administered to the patient once per day or twice per day.

In some embodiments, the present invention provides a unit dosage form comprising a composition comprising:
(a) X4P-001, or a pharmaceutically acceptable salt thereof—about 10-30% by weight of the composition;
(b) microcrystalline cellulose—about 60-80% by weight of the composition;
(c) croscarmellose sodium—about 5-10% by weight of the composition;
(d) sodium stearyl fumarate—about 0.5-2% by weight of the composition; and
(e) colloidal silicon dioxide—about 0.1-1.0% by weight of the composition.

In some embodiments, the present invention provides a unit dosage form comprising a composition comprising:
(a) X4P-001, or a pharmaceutically acceptable salt thereof—about 14.7% by weight of the composition;
(b) microcrystalline cellulose—about 78.1% by weight of the composition;
(c) croscarmellose sodium—about 6.0% by weight of the composition;
(d) sodium stearyl fumarate—about 1.0% by weight of the composition; and
(e) colloidal silicon dioxide—about 0.2% by weight of the composition.

In some embodiments, the present invention provides a unit dosage form comprising a composition comprising:
(a) X4P-001, or a pharmaceutically acceptable salt thereof—about 10-20% by weight of the composition;
(b) microcrystalline cellulose—about 25-40% by weight of the composition;
(c) dibasic calcium phosphate dihydrate—about 35-55% by weight of the composition;
(d) croscarmellose sodium—about 4-15% by weight of the composition;
(e) sodium stearyl fumarate—about 0.3-2% by weight of the composition;
(f) colloidal silicon dioxide—about 0.1-1.5% by weight of the composition; and
(g) sodium lauryl sulfate—about 0.1-1.5% by weight of the composition.

In some embodiments, the present invention provides a unit dosage form comprising a composition comprising:
(a) X4P-001, or a pharmaceutically acceptable salt thereof—about 12.85% by weight of the composition;
(b) microcrystalline cellulose—about 31.92% by weight of the composition;
(c) dibasic calcium phosphate dihydrate—about 44.4% by weight of the composition;
(d) croscarmellose sodium—about 8.33% by weight of the composition;
(e) sodium stearyl fumarate—about 1.38% by weight of the composition;
(f) colloidal silicon dioxide—about 0.42% by weight of the composition; and
(g) sodium lauryl sulfate—about 0.7% by weight of the composition.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

### Example 1: Non-Clinical Evaluation of X4P-001 Effects on CXCR4

### In Vitro Pharmacology

The in-vitro pharmacology of X4P-001 (formally designated AMD11070) was extensively studied and the results reported [Mosi 2012]. Presented below is the relevant information from the Mosi 2012 literature publication. The SDF-1α isoform was used for the experiments described below.

### X4P-001 Inhibition of SDF-1α Binding to CXCR4

X4P-001 was shown to inhibit binding of [¹²⁵I]-SDF-1α to CCRF-CEM cells (T-lymphoblastoid cell line which naturally express CXCR4 [Crump 1997]) in a heterologous competition binding assay. The results of the assay are shown in FIG. 2 below. The data was fitted to a single site binding model and gave an IC₅₀ of 12.5±1.3 nM.

### X4P-001 Inhibition of CXCR4 Cell Signaling

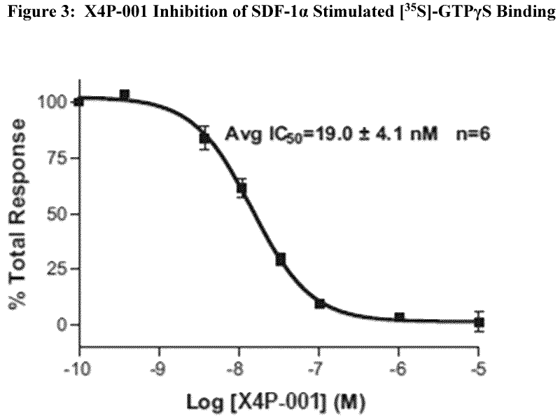

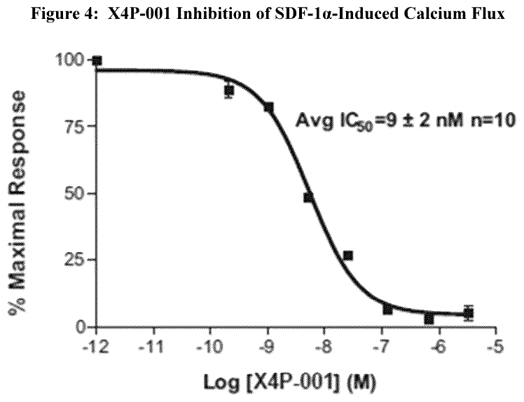

CXCR4 is a G-protein coupled receptor [Baggiolini 1998, Zlotnik 2000]. As such the activation of the receptor can be measured using a nonhydrolysable analogue of GTP such as fluorescently labeled Europium-GTP (Eu-GTP) or radio labeled [³⁵S]-GTPyS. The results shown in FIG. 3 and FIG. 4 showed that X4P-001 inhibited CXCR4 activation with IC₅₀ values of 39.8±2.5 nM and 19.0±4.1 nM in the Eu-GTP binding and [³⁵S]-GTPγS assays, respectively.

Upon activation of a G-protein coupled receptor, intracellular signaling pathways are triggered resulting in the release of calcium from intracellular stores. This calcium flux can be assayed using a calcium-chelating molecule, Fluo-4, which fluoresces upon binding calcium. X4P-001 was able to inhibit SDF-1α (2.5 nM SDF-1α) mediated calcium flux in CCRF-CEM cells with an IC₅₀ of 9.0±2.0 nM. The result is shown in FIG. 5.

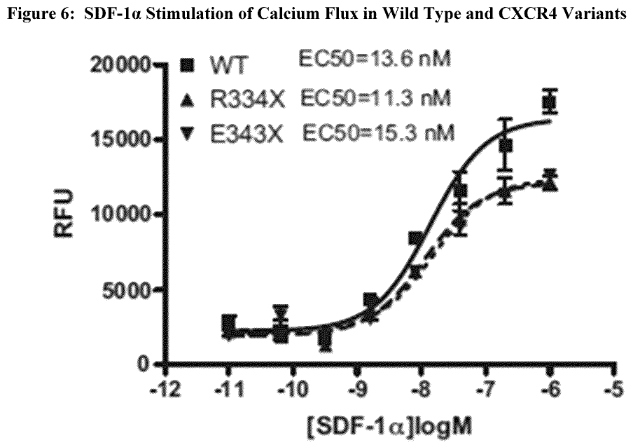

A key property of all chemokines is that they induce a chemotactic response to a chemokine concentration gradient. X4P-001 was able to inhibit SDF-1α mediated chemotaxis of CCRF-CEM cells with an IC₅₀ of 19.0±4.0 nM as shown in FIG. 6.

A summary of the above in vitro results is presented in Table 1 below:

TABLE 1
In Vitro Concentrations of X4P-001(IC₅₀) Associated
with Different Biological Responses

| Response | IC₅₀ (nM)^{a} |
|---|---|
| Ligand Binding | 12.5 ± 1.3 |
| Eu-GTP | 39.8 ± 2.5 |
| [³⁵S]-GTP | 19.0 ± 4.1 |
| Calcium Flux | 9.0 ± 2.0 |
| Chemotaxis | 19.0 ± 4.0 |
| Average IC₅₀ | 21.5 |

^{a}= Results are expressed as mean ± SE

### X4P-001 Selectivity for CXCR4

In order to demonstrate the specificity of X4P-001 for CXCR4 it was tested in calcium signaling assays against a panel of chemokine receptors, and in ligand binding assays for BLT₁, the receptor for leukotriene B4 (LTB₄), and CXCR7. LTB₄ is a potent chemoattractant and its receptor is a G-protein coupled receptor. The results in Table 2 show that the IC₅₀ of X4P-001 against CCR1, CCR2b, CCR4, CCR5, CCR7, CXCR3, and LTB₄ was >50 mM in all cases. X4P-001 did not inhibit SDF-1α binding to CXCR7 at a concentration of 10 mM, the maximum concentration tested in this assay. Together these data indicate that X4P-001 is a selective inhibitor of CXCR4.

In order to demonstrate the specificity of X4P-001 for CXCR4 it was tested in calcium signaling assays against a panel of chemokine receptors, and in ligand binding assays for BLT₁, the receptor for leukotriene B4 (LTB₄), and CXCR7. LTB₄ is a potent chemoattractant and its receptor is a G-protein coupled receptor. The results in Table 2 show that the IC₅₀ of X4P-001 against CCR1, CCR2b, CCR4, CCR5, CCR7, CXCR3, and LTB₄ was >50 mM in all cases. X4P-001 did not inhibit SDF-1α binding to CXCR7 at a concentration of 10 mM, the maximum concentration tested in this assay. Together these data indicate that X4P-001 is a selective inhibitor of CXCR4.

TABLE 2
Calcium Flux Response for Cell Lines Treated
with X4P-001 for IC₅₀ Determination

| | | | IC₅₀AMD11070 |
|---|---|---|---|
| Receptor | Cell line | Ligand | (μM) |
| CCR1 | HEK293F-CCR1 | MIP-1α/CCL3 | >50 |
| CCR2b | HEK293F-CCR2b | MCP-1/CCL2 | >50 |
| CXCR3 | HEK293F-CXCR3- | IP-10/CXCL10 | >50 |
| | Gαq15 | | |
| CXCR7 | Cf2Th.CXCR7 | SDF-1α/CXCL12 | >10 |
| CCR4 | HEK293F-CCR4- | TARC/CCL17 | >50 |
| | Gαq15 | | |
| CCR5 | HEK293F-CCR5 | RANTES/CCL5 | >50 |
| CCR7 | CCRF-CEM | MIP-3β/CCL19 | >50 |
| BLT₁ | CHO-S-LTB₄ | LTB₄ | >50 |

### X4P-001 Inhibition of C-Terminal Variants of CXCR4

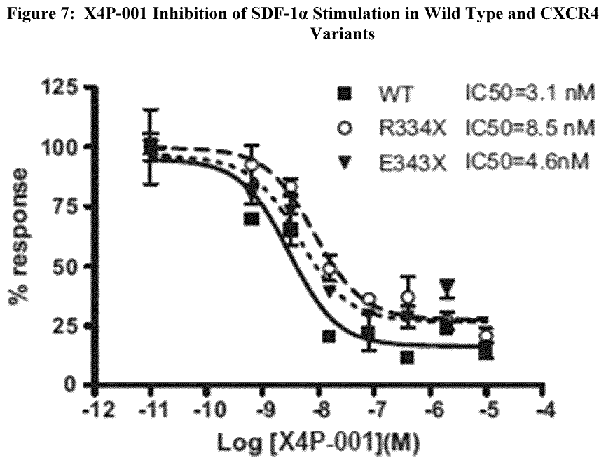

From a therapeutic perspective it is important that CXCR4 antagonists can act on CXCR4 variants. Carboxy-terminal truncated variants of CXCR4 have been reported associated with WHIM syndrome; nonsense mutations resulting in a 19 amino acid truncation (R334X), and a 10 amino acid truncation (E343X) and a frameshift mutation resulting in a 13 amino acid truncation (S339fs342X) [Hernandez 2003, Kawai 2009]. The R334X and E343X CXCR4 variants were cloned and transiently expressed in the canine thymus cell line Cf2Th. This cell line was chosen due to its lack of expression of CXCR4 [Wong 2008]. Wild type CXCR4 was similarly sub-cloned into this cell line for control studies. In control studies it was demonstrated that both these carboxy-terminal truncated variants were able to respond to SDF-1α in the calcium flux assay with a similar potency to wild type CXCR4. The EC₅₀ values for SDF-1α were 13.6, 11.3 and 15.3 nM against the wild type, R334X and E343X variants of CXCR4, respectively (FIG. 6). The inhibitory effect of X4P-001 on SDF-1α-mediated calcium flux was assessed for the two CXCR4 variants. Both variants were inhibited to a similar extent as the wild type CXCR4 with IC₅₀ values of 3.1, 8.5 and 4.6 nM for the wild type, R334X and E343X variants respectively (FIG. 7).

Discussion and Conclusions from In Vitro Studies

Using the CCRF-CEM cell line, which naturally expresses CXCR4 [Crump 1997] it was shown that X4P-001 inhibits SDF-1α ligand binding to CXCR4 with an IC₅₀ of 12.5±1.3 nM. X4P-001 also inhibited CXCR4 activation and signaling as shown by inhibition of SDF-1α mediated G-protein activation of the CXCR4 receptor in two assays using either the fluorescent Eu-GTP or the radiolabeled [³⁵S]-GTPγS binding assays with IC₅₀ values of 39.8±2.5 nM and 19.0±4.1 nM, respectively, and inhibition of SDF-1α mediated calcium flux with an IC₅₀ of 9.0±2.0 nM. X4P-001 also inhibited SDF-1α-mediated chemotaxis, a CXCR4-mediated physiological response, with an IC₅₀ of 19.0±4.0 nM. In addition, X4P-001 had little or no inhibitory effect on either MIP1α, MCP-1, TARC, RANTES, MIP-3β, or IP10 mediated calcium flux, ligands for CCR1, CCR2b, CCR4, CCR5, CCR7 and CXCR3, respectively, or SDF-1α binding to CXCR7, or LTB4 binding to BLT1, an alternative G-protein coupled receptor that mediates chemotaxis. These data indicate that X4P-001 is a selective inhibitor of CXCR4 over the other chemokine receptors evaluated.

Mutations in CXCR4 resulting in truncation of the intracellular carboxy-terminus of the receptor have been linked to the rare condition, WHIM syndrome [Hernandez 2003, Kawai 2009]. Two of these CXCR4variants were evaluated and the results demonstrated that X4P-001 was able to inhibit SDF-1α-mediated calcium flux in these carboxy-terminal truncated variants. These data further indicate that X4P-001 acts via interaction with the extracellular region of CXCR4. Furthermore it is significant from the perspective of X4P-001 as a potential therapeutic option for WHIM syndrome that it can inhibit multiple variants of CXCR4.

Additionally it was shown that X4P-001 is an allosteric inhibitor of CXCR4 by comparing the dose/response of SDF-1α in the calcium flux assay in the presence of increasing amounts of X4P-001 [Mosi 2012]. Based on inhibition being mediated by non-competitive binding, the extent of inhibition is therefore dependent solely on the concentration of X4P-001 and is independent of the concentration of SDF-1α ligand. In-Vivo Pharmacology

The primary in vivo pharmacologic effect of X4P-001 is mobilization of white blood cells (WBC) from bone marrow. Three studies are summarized below which demonstrate the mobilization of WBC from the bone marrow of beagle dogs and C3W/He J mice.

### Hematologic Effects in the Male Beagle Dog

Three fasted male Beagle dogs received a single dose of X4P-001 in aqueous solution by oral gavage at dose levels of 5, 15, and 35 mg/kg (1 dog per dose level) in a volume of 1 mL/kg. Blood samples (approximately 3 mL each) were obtained at multiple timepoints from each animal by direct venipuncture of the jugular vein and collected using Vacutainer® tubes containing K₃EDTA as the anticoagulant. Blood samples were obtained at pre-dose, and 0.25, 0.5, 0.75, 1, 1.5, 2, 3, 4, 7, 12, and 24 hours post-dose. Blood samples were stored at ambient room temperature prior to automated differential analysis

Body weights were determined prior to dosing on the day of test article administration. Animals were observed at least once daily and at times of blood sampling.

Hematology parameters included the following:
White Blood Cell Count (WBC)
Differential white blood cell count (absolute and relative)
Neutrophil
Lymphocytes
Monocytes
Eosinophils
Basophils
Large Unstained Cells (LUC)
Hematocrit (HCT)
Hemoglobin (HGB)
Mean Corpuscular Hemoglobin (MCH)
Mean Corpuscular Hemoglobin Concentration (MCHC)
Mean Corpuscular Volume (MCV)
Platelet Count (PLT)
Red Blood Cell Count (RBC)

### Results

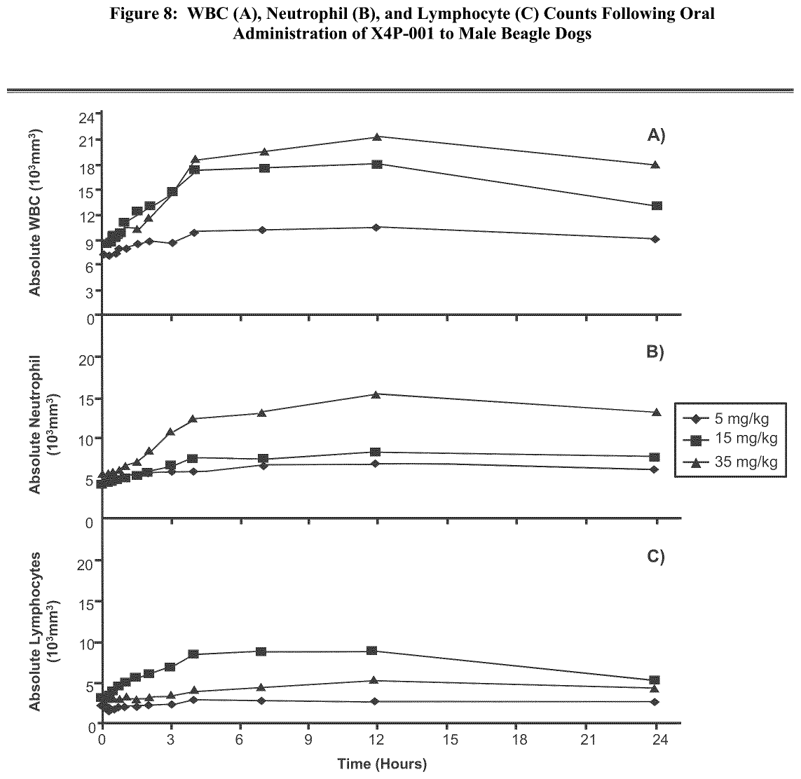

The effect of X4P-001 on WBC and absolute neutrophil and lymphocyte counts is shown in FIG. 8. Maximal increases in WBC occurred 4-12 hours post-dose. Peak elevations ranged from 1.8-2.9-fold above baseline values at the 15 and 35 mg/kg_dose levels, with somewhat lower (1.5-fold) elevations observed at the 5 mg/kg dose_level. Although limited by the small sample size, these results suggest that maximal_increases may have been achieved at the higher dose levels. WBC, neutrophil, and_lymphocyte counts remained elevated at the 15 and 35 mg/kg dose levels at 24 hours, with evidence of return to baseline. No other hematological effects were observed.

A 28-Day Oral (Capsule) Study in the Beagle Dog with a 14-Day Recovery Period

A 28-Day GLP oral (capsule) toxicology study was conducted with X4P-001 in the male and female beagle dog, and hematology effects were observed, with X4P-001 administered twice-daily (at least 7 hours apart) by oral capsule for 28 days. A subset of treated animals was evaluated after a 14-day recovery period. Table 3 presents the protocol design and Table 4 the evaluations schedule.

TABLE 3
Protocol Design for 28-Day Toxicity Study in the Dog

| | | Animals | |
|---|---|---|---|
| | Dose Level | Terminal | Animals |
| Group | (mg/kg/day)^{a} | Necropsy | 14-day Recovery |
| 1 | 0 (empty capsule) | 3 M, 3 F | 2 M, 2 F |
| 2 | 10 | 3 M, 3 F | — |
| 3 | 30 | 3 M, 3 F | — |

TABLE 4
Protocol Evaluations and Schedules

| Evaluations | Schedule |
|---|---|
| Study duration | Days −10 through Day 42 |
| Treatment | Days 1 through 28, twice daily |
| Clinical | Twice daily |
| observation | |
| Food | Daily |
| consumption | |
| Body weight | Weekly |
| Vital signs^{a} | Predose acclimation period; final |
| | dosing week; final recovery week |
| Ophthalmology | Predose and during Week 4 |
| Electrocardiogram | Predose and during Week 4, at ~1 |
| evaluation | hour post-first daily dose |
| Clinical | Predose d −10, d −2; Post-dose, Day |
| Pathology^{b} | 29 (all groups), Day 42 |
| | (recovery only) |
| Necropsy^{c} | Day 29, terminal; Day 42, recovery |

^{a}Vital signs comprise heart rate, blood pressure, and body temperature
^{b}Clinical pathology comprised hematology, coagulation, serum, and urinalysis (done only once predose).
^{c}Necropsy studies comprise organ weight, macroscopic, and microscopic observations, including 500-cell bone marrow differential count.

As shown in Table 5 below, increases in absolute counts for neutrophils, lymphocytes, and monocytes were observed at termination (Day 28); these were of greater magnitude and more likely statistically significant in females. These changes were considered consistent with the pharmacological effects of X4P-001. After the 14-day recovery period (only 100 mg/kg dose group evaluated) all hematology results returned to within normal levels.

TABLE 5
Hematology Findings at Termination in
28-Day Oral Toxicity Study in the Dog

| | 10 mg/kg/d | 30 mg/kg/d | 100 mg/kg/d |
|---|---|---|---|
| Observation | (3M, 3F) | (3M, 3F) | (3M, 3F) |
| Hematology | M incr 1.2x; | M incr 1.2x; | M incr 1.8x; |
| neutrophils | F incr 1.9x† | F incr 2.3x† | F incr 2.8x† |
| (abs) | | | |
| lymphocytes | M incr 1.3x; | M incr 1.6x; | M incr 2.3x†; |
| (abs) | F incr 1.4x | F incr 1.6x† | F incr 1.4x† |
| monocytes (abs) | M incr 1.2x; | M incr 1.3x; | M incr 1.9x†; |
| | F incr 1.6x† | F incr 1.9x† | F incr 2.4x† |
| reticulocytes | No changes | No changes | F decr 0.24x† |
| Coagulation | No changes | No changes | No changes |

abs, absolute;
†p < 0.05 compared with control animals of the same sex

### Hematologic Effects of X4P-001 in Mice

A further study was conducted to determine whether X4P-001 mobilizes progenitor/stem cells in mice. All experiments were performed in C3W/He J mice. X4P-001 and AMD3100/plerixafor were administered via single subcutaneous injection at the doses described below. The mobilization capacity of X4P-001 was assessed by the numbers of granulocyte-macrophage (CFU-GM), erythroid (BFU-E) and multipotential (CFU-GEMM) progenitor cells per mL of blood. The progenitors were stimulated to form colonies in vitro with the combination of 1U/mL rhu EPO, 50 ng/ml rmu SLF, 5% vol/vol pokeweed mitogen mouse spleen cell conditioned medium (PWMSCM), and 0. 1 mM hemin. Plates were scored 7 days after incubation at 37° C., 5% CO₂, lowered (5% CO₂) and in a humidified chamber.

### Results

X4P-001 mobilized progenitors in C3H/HeJ mice following a single subcutaneous injection. In the first experiment (data shown in Table 6), mice received a dose of 5 mg/kg- and the number of progenitors in the circulating blood was measured at various time points (0.25, 0.5, 1, 2, 6 and 24 hours). The peak of nucleated cell mobilization occurred at approximately 1-2 hours post-injection. Peak increases of CFU-GM, BFU-E and CFU-GEMM were 4.21 (30 min.), 2.49-2.54 (30-60 min.), and 2.58-2.67 (30-60 min.)-fold, respectively over control (saline injection).

TABLE 6
X4P-001 Time Course of Progenitor Mobilization

| | | | AMD11070 Time Course (dose = 5 mg/kg) | | | | | |
|---|---|---|---|---|---|---|---|---|
| | | Control | @-15” | @-30” | @-60” | @-2’ | @-6’ | @-24’ |
| Nucleated | Mean | 4.35 | 5.10 | 6.14 | 6.92 | 8.29 | 5.66 | 4.31 |
| Cellularity | STD | 0.14 | 1.09 | 1.20 | 0.57 | 0.55 | 0.28 | 0.82 |
| (× 10⁶/mL) | STE | 0.08 | 0.63 | 0.69 | 0.33 | 0.32 | 0.16 | 0.47 |
| PBL-LD | Fold | 1.00 | 1.17 | 1.41 | 1.59 | 1.90 | 1.30 | 0.99 |
| | Chg | | | | | | | |
| | P | 1.000 | 0.307 | 0.062 | 0.002 | 0.000 | 0.002 | 0.930 |
| GM | Mean | 302.3 | 785.1 | 1273.8 | 866.0 | 897.5 | 387.5 | 386.3 |
| | STD | 20.5 | 180.3 | 85.4 | 197.8 | 165.6 | 54.6 | 110.5 |
| | STE | 11.8 | 104.1 | 49.3 | 114.2 | 95.6 | 31.5 | 63.8 |
| | Fold | 1.00 | 2.60 | 4.21 | 2.86 | 2.97 | 1.28 | 1.28 |
| | Chg | | | | | | | |
| | P | 1.000 | 0.010 | 0.000 | 0.008 | 0.003 | 0.065 | 0.265 |
| BFU | Mean | 92.5 | 148.8 | 230.4 | 235.1 | 165.3 | 99.9 | 84.6 |
| | STD | 30.9 | 27.1 | 70.2 | 68.2 | 47.5 | 17.8 | 44.4 |
| | STE | 17.8 | 15.6 | 40.5 | 39.4 | 27.4 | 10.3 | 25.7 |
| | Fold | 1.00 | 1.61 | 2.49 | 2.54 | 1.79 | 1.08 | 0.92 |
| | Chg | | | | | | | |
| | P | 1.000 | 0.076 | 0.036 | 0.030 | 0.090 | 0.735 | 0.814 |
| GEMM | Mean | 38.6 | 65.6 | 99.6 | 103.1 | 68.9 | 37.6 | 37.7 |
| | STD | 10.6 | 17.6 | 24.2 | 20.3 | 23.7 | 16.0 | 20.6 |
| | STE | 6.1 | 10.2 | 14.0 | 11.7 | 13.7 | 9.3 | 11.9 |
| | Fold | 1.00 | 1.70 | 2.58 | 2.67 | 1.78 | 0.97 | 0.98 |
| | Chg | | | | | | | |
| | P | 1.000 | 0.085 | 0.016 | 0.008 | 0.114 | 0.934 | 0.946 |

Animals per group = 3,
control group = 1,
total animals = 21

An X4P-001 dose-response was performed by measurement of the number of circulating progenitors in the blood at 1 hour post-injection at various doses (1.5, 2.5, 5, 10 and 20 mg/kg). As shown in Table 7, there appears to be an upper limit to the number of progenitors that can be mobilized with X4P-001, exemplified by the fold increases of CFU-GM. The numbers of CFU-GM in the circulating blood dose-dependently increased with peak fold increase of 6.0-7.7 over control at 5-20 mg/kg. Peak fold increases respectively of 2.3 and 3.8 for BFU-E and CFU-GEMM were noted at 10 mg/kg. At doses below 5 mg/kg X4P-001, the fold-increases in the numbers of BFU-E and CFU-GEMM were not statistically significant.

TABLE 7
Dose Response in C3H/HeJ Mice

| | | | AMD11070 (mg/kg) | | | | |
|---|---|---|---|---|---|---|---|
| | | Control | 20 | 10 | 5 | 2.5 | 1.5 |
| Nucleated Cellularity | Mean | 6.48 | 9.62 | 9.94 | 7.65 | 8.29 | 6.94 |
| (× 10⁶/mL) | STD | 0.69 | 1.26 | 4.02 | 2.74 | 2.07 | 0.50 |
| PBL-LD | STE | 0.40 | 0.73 | 2.32 | 1.58 | 1.20 | 0.29 |
| | Fold | 1.00 | 1.48 | 1.53 | 1.18 | 1.28 | 1.07 |
| | Chg | | | | | | |
| | P | 1.000 | 0.019 | 0.216 | 0.514 | 0.225 | 0.406 |
| GM | Mean | 188.0 | 1314.2 | 1444.2 | 1119.8 | 626.5 | 428.0 |
| | STD | 51.8 | 262.0 | 939.8 | 1011.9 | 220.4 | 118.7 |
| | STE | 29.9 | 151.2 | 542.6 | 584.2 | 127.3 | 68.5 |
| | Fold | 1.0 | 7.0 | 7.7 | 6.0 | 3.3 | 2.3 |
| | Chg | | | | | | |
| | P | 1.000 | 0.002 | 0.082 | 0.186 | 0.028 | 0.033 |
| BFU | Mean | 114.4 | 261.4 | 268.1 | 181.6 | 144.8 | 143.8 |
| | STD | 5.6 | 35.8 | 61.4 | 58.6 | 79.3 | 47.1 |
| | STE | 3.2 | 20.7 | 35.5 | 33.8 | 45.8 | 27.2 |
| | Fold | 1.0 | 2.3 | 2.3 | 1.6 | 1.3 | 1.3 |
| | Chg | | | | | | |
| | P | 1.000 | 0.002 | 0.012 | 0.119 | 0.544 | 0.343 |
| GEMM | Mean | 58.4 | 145.0 | 224.4 | 141.0 | 78.3 | 53.3 |
| | STD | 45.5 | 50.5 | 60.7 | 34.4 | 8.1 | 8.9 |
| | STE | 26.3 | 29.2 | 35.0 | 19.8 | 4.7 | 5.1 |
| | Fold | 1.0 | 2.5 | 3.8 | 2.4 | 1.3 | 0.9 |
| | Chg | | | | | | |
| | P | 1.000 | 0.092 | 0.019 | 0.066 | 0.498 | 0.857 |

Animals per group = 3,
control group 1,
total animals = 18

A final experiment was performed to compare the progenitor cell mobilization capacity of X4P-001 and AMD3100/plerixafor. Both drugs were administered subcutaneously at a dose of 5 mg/kg, and the number of progenitors in the circulating blood were measured for AMD3100 at a single 1 hour time point (the peak of mobilization with AMD3100, data not shown) versus X4P-001 at 0.25, 0.5, 1 and 2 hours post-injection. As shown in Table 8 comparing the fold-increase in CFU-GM, BFU-E, and CFU-GEMM, AMID3100 caused respective maximum increases of 9.11, 3.12, and 4.35, whereas respective peaks of mobilization with X4P-001 were 3.56, 2.84 and 3.21.

TABLE 8
X4P-001 Time Course Compared to ADM3100/Plerixafor (Dose 5 mg/kg)

| | | | AMD3100 | AMD11070 | | | |
|---|---|---|---|---|---|---|---|
| | | Control | @-60” | @-15” | @-30” | @-60” | @-2’ |
| Nucleated Cellularity | Mean | 6.23 | 10.08 | 8.04 | 8.28 | 7.34 | 9.71 |
| (×10⁶/mL) | STD | 2.16 | 2.13 | 1.30 | 0.94 | 0.69 | 1.29 |
| PBL-LD | STE | 1.25 | 1.23 | 0.75 | 0.54 | 0.40 | 0.74 |
| | Fold | 1.00 | 1.62 | 1.29 | 1.33 | 1.18 | 1.56 |
| | Chg | | | | | | |
| | P | 1.000 | 0.092 | 0.281 | 0.205 | 0.444 | 0.074 |
| GM | Mean | 214.1 | 1950.3 | 588.3 | 705.9 | 761.4 | 619.6 |
| | STD | 118.2 | 566.4 | 168.1 | 151.5 | 239.2 | 158.7 |
| | STE | 68.2 | 327.0 | 97.1 | 87.5 | 138.1 | 91.6 |
| | Fold | 1.00 | 9.11 | 2.75 | 3.30 | 3.56 | 2.89 |
| | Chg | | | | | | |
| | P | 1.000 | 0.007 | 0.034 | 0.011 | 0.024 | 0.024 |
| BFU | Mean | 66.5 | 207.7 | 188.9 | 151.9 | 144.3 | 108.5 |
| | STD | 39.6 | 35.4 | 55.0 | 23.8 | 47.5 | 43.0 |
| | STE | 22.9 | 20.4 | 31.7 | 13.8 | 27.4 | 24.8 |
| | Fold | 1.00 | 3.12 | 2.84 | 2.29 | 2.17 | 1.63 |
| | Chg | | | | | | |
| | P | 1.000 | 0.010 | 0.035 | 0.033 | 0.095 | 0.281 |
| GEMM | Mean | 31.8 | 138.5 | 93.8 | 79.0 | 102.2 | 62.4 |
| | STD | 2.6 | 18.1 | 21.1 | 34.5 | 50.5 | 34.9 |
| | STE | 1.5 | 10.5 | 12.2 | 19.9 | 29.1 | 20.1 |
| | Fold | 1.00 | 4.35 | 2.95 | 2.48 | 3.21 | 1.96 |
| | Chg | | | | | | |
| | P | 1.000 | 0.001 | 0.007 | 0.078 | 0.074 | 0.205 |

Animals per group = 3,
control group = 1,
total animals = 18

Conclusions from In Vivo Studies

Single oral doses of X4P-001 at 5, 15, and 35 mg/kg in beagle dogs resulted in increased levels of total circulating WBC, neutrophils, and lymphocytes. The increases were consistently apparent at 4 hours and typically peaked at 12 hour, occasionally earlier. At 5 mg/kg, all three cell counts increased to 1.47×baseline. At 15 mg/kg, neutrophils increased to 1.8× and lymphocytes to 2.9×; and at 35 mg/kg, neutrophils to 2.7× and lymphocytes to 1.9×.

In multiple-dose toxicity studies in dogs, hematological effects after 28 days were qualitatively and quantitatively consistent with the findings in the single dose study in beagle dogs.

In C3H/HeJ mice, X4P-001 dose-dependently increased the number of circulating progenitors up to a dose of 5-10 mg/kg s.c.

### Example 2: Clinical: Patients to be Treated

Patients who may be treated according to the present invention include patients who have been diagnosed with WHIMS or with MKX; and patients who present the characteristic mutations in their CXCR4 gene. Other patients who may benefit from the present invention may include individuals presenting with the following screening criteria:

Neutropenia (ANC≤400 or ≤600/μL) and or lymphopenia (ALC≤650 or ≤1000/μL)—the latter is not characteristic of other chronic neutropenias;
Neutropenia and chronic warts;
Myelokathexis on bone marrow aspirate;
Patients meeting the above criteria are genetically screened for MKX. Patients with the characteristic mutations in CXCR4 are the most likely to benefit from treatment in accordance with the present invention.

Thus, the effects of X4P-001 is expected to be greatest in patients with myelokathexis associated with mutation in CXCR4. In addition, patients who may benefit from treatment according to the present invention include individuals presenting with the following screening criteria:
1. Has a genotype-confirmed CXCR4 mutation consistent with WHIM syndrome; and
2. Has ANC≤400 or ≤600/μL, or ALC≤650 or ≤1000/μL, or both, on at least two independent blood samples collected over a period of up to 14 days.
3. Have one of the following findings:
   A bone marrow aspirate or biopsy showing myelokathexis
   Peripheral WBC counts (≥2 independent samples, obtained in the absence of signs or symptoms of acute infection, and when not having received G- or GM-CSF in the past 7 days) showing absolute neutrophil count <900/μL and/or absolute lymphocyte count <1,500/μL.

Examples of candidate endpoints based on 6 months on-treatment compared to the prior 6 months without treatment include:
50% reduction in hospitalizations
50% reduction in infections requiring courses of systemic antibiotics
50% reduction in area involving cutaneous warts
sustained increases in circulating neutrophils (e.g., ANC>600/μL; ANC>800/μL; ANC>1000/μL; or ANC>1,200/μL on at least 85% of assessments)
sustained increases in circulating lymphocytes (e.g., ALC>1000/μL; ALC>1,200/μL; or ALC>1,500/μL on at least 85% of assessments)
Achieve pre-defined levels of protective antibody in response to at least 2 approved vaccines previously administered without achieving that level.
50% reduction in days of work or school missed due to infection
sustained increases in circulating neutrophils.

Not all endpoints are applicable to all patients, just as all patients with WHIM do not exhibit identical clinical manifestations. However, all patients exhibit at least one clinical and one laboratory metric.

Patients are preferably initiated on treatment orally with X4P-001 25 mg once daily, 25 mg twice daily, or 50 mg once daily. Provision is made for dose reduction (which can be via increased interval; e.g., to every other day or twice weekly) in the event of toxicity or dose increase (e.g., to >50 mg once daily or higher daily dosage, such as 100 mg/day or 150 mg/day) in the event of an inadequate response.

An exemplary initial dosage is via X4P-001 25 mg capsules, administered orally in the morning in a fasted state, with no food or drink (except water) after midnight and continuing until 2 hr post-dose. In twice daily dosage regimens, capsules are preferably administered orally twelve hours apart.

### Example 3: Clinical Treatment Regimens

Dosing Regimen for Patients with WHIM Syndrome:

X4P-001 at a determined dose of 25 mg or 50 mg daily is administered orally. Patients are instructed about both dosing schedule and requirements relating to food or drink near the time of dosing.

Dosing Schedule. The first daily dose is taken in the morning. For twice daily dosing, doses should be taken twelve hours apart. Dosing should be at the same times each day±2 hr.

Restrictions relating to food. Absorption is impacted by food and patients will be instructed as follows:

For the morning dose:
No food or drink (except water) after midnight until the time of dosing
No food or drink (except water) for 2 hour after dosing.

Dosing of X4P-001 may be adjusted by the clinician as appropriate. The dose of X4P-001 may be lowered according to the judgment of the clinician. If a patient receiving X4P-001 experiences an adverse event at Grade>2, the dose of X4P-001 may be lowered according to the judgment of the clinician. If a patient successfully completes the first 2 to 4 weeks of treatment, that is, without experiencing any adverse events greater than Grade 2, the daily dose of X4P-001 may be increased consistent with the judgment of the clinician.

Alternative Dosing Regimens for Patients with WHIM Syndrome

Patients' initial absolute neutrophil count (ANC) (neutropenia) (ANC<400 or <600/μL) and/or absolute lymphocyte count (ALC) (lymphopenia) (ALC<650_or <1000/μL)] are measured. [Note—the latter is not characteristic of other chronic neutropenias]. If the patient exhibits ANC below 400 or below 600/μL; and/or ALC remains below 650 or below 1000/μL, then treatment with X4P-001 is initiated. Patients are initiated on treatment with X4P-001 25 mg orally once daily, 25 mg orally twice daily, or 50 mg orally once daily. Provision is made for dose reduction (which can be via increased interval, e.g., to every other day or twice weekly) or halt of administration in the event of toxicity, or dose increase (e.g., to >50 mg daily or higher daily dosage) in the event of an inadequate response.

ANC and ALC are monitored monthly or, preferably, bi-weekly. If ANC>400 or >600/μL; and/or ALC>650 or >1000/μL is achieved, the patient will continue on the original daily dosage regimen. If ANC remains below 400 or below 600/μL; and/or ALC remains below 650 or below 1000/μL, and the patient exhibits no severe adverse effects, the patient's dose will be increased to by 25 mg or 50 mg daily [or by 25 mg orally twice daily; 12 hours apart].

Patients on the increased dose of 50 mg/μL per day will continue to be monitored monthly or, preferably, bi-weekly. If ANC≥400 or ≥600/μL; ALC≥650 or ≥1000/μL is achieved (without severe adverse effects), the patient will continue on the increased dosage regimen. If ANC remains below 400 or below 600/μL; and/or ALC remains below 650 or below 1000/μL, (and patient exhibits no severe adverse effects), the patient's dose will be further increased to by an additional 25 mg or 50 mg daily.

The above procedures of increasing daily dosage regimens may be repeated until the patient achieves ANC≥400 or ≥600/μL; and/or ALC≥650 or ≥1000/μL (without severe adverse effects); or until the patient is being treated at a maximum tolerated daily dose.

Alternatively, ANC and ALC are analyzed as area-under-the-curve (AUC) relative to pre-specified clinically meaningful thresholds of 400 or 600/μL and 650 or 1000/μL, respectively. The 24-hour AUC will be calculated using the trapezoidal method with area above threshold being positive, and area below threshold, negative. Patients with AUC_{ANC} <2000 cell·hr/u1 or AUC_{ALC} <5000 cell·hr/μL at monthly or bi-weekly evaluations will have X4P-001 daily dose increased in 25 mg or 50 mg increments up to a maximum dose of 150 mg QD. Because ANC and ALC in WHIM patients are significantly impacted by acute infection, alone or with antibiotic, G-CSF or IVIG treatment, monitoring of AUC should be delayed or discontinued in patients with acute infection, until such patient has remained afebrile for at least 2 weeks.

If the patient experiences adverse effects at any time, provision is made for dose reduction (i.e., lower dosage and/or increased interval between administrations drug), or administration is halted. Additionally, the treating physician may use his or her professional judgment and discretion in determining the starting dose, and how best to titrate to the appropriate dose of X4P-001 for any individual patient.

An exemplary composition of a X4P-001 25 mg capsule that may be used is shown in Table 9 below.

TABLE 9
Quantitative Composition of Exemplary X4P-001 25 mg Capsule

| | Reference | | Quantity | |
|---|---|---|---|---|
| Component | to Standard | Function | (mg/capsule) | % w/w |
| X4P-001 | In House | Active | 25.0 | 14.7 |
| | | Ingredient | | |
| Microcrystalline | NF | Diluent | 132.7 | 78.1 |
| Cellulose | | | | |
| Croscarmellose | NF | Disintegrant | 10.2 | 6.0 |
| Sodium | | | | |
| Sodium Stearyl | NF | Lubricant | 1.7 | 1.0 |
| Fumarate | | | | |
| Colloidal Silicon | USP | Flow Aid | 0.4 | 0.2 |
| Dioxide | | | | |
| Sum Total | | | 170.0 | 100.0 |
| Hard Gelatin | USP | Packaging | NA | NA |
| Capsules, Size 1 | | | | |

### Example 4: Assessments of Treatment Effect

### Circulating White Blood Cells

### Whole Blood Samples are Analyzed for:

CBC and absolute leukocyte differential counts by standard laboratory methods, including WBC counts, including absolute numbers of lymphocytes, neutrophils, and CD34+ cells. The number and percentage of patients achieving ANC>1,500/μL; ALC>900/μL. The absolute increase in blood neutrophil counts from pre-treatment baseline for each subject, including at the maximum observed in the hours post-dosing; and the maximum observed pre-dose on stable drug administration regimen. These results are compared with data from healthy adults administered X4P-001.

Peripheral Blood Mononuclear Cells (PBMC) subpopulations by flow cytometry are shown below in Table 10.

TABLE 10
Candidate Subsets of Circulating Lymphocytes and Monocytes

| | | |
|---|---|---|
| CD4+ T cells | CD3− CD56+ (NK cells) | CD34+ (stem cells) |
| CD4+ CD45RA+ | CD19+ (B cells) | CD49f+ (stem cells) |
| (naïve T cells) | CD19+ CD27− IgM+ | CD90+ (stem cells) |
| CD4+ CD45RA− | (transitional B cells) | |
| (memory T cells) | CD14+ (monocytes) | |
| CD8+ T cells | CD14+ CD16− | |
| CD8+ CD45RA+ | (classical monocytes) | |
| (naïve T cells) | CD14+ CD16+ | |
| CD8+ CD45RA− | (inflammatory monocytes) | |
| (memory T cells) | | |

### Immunoglobulins and Specific Antibodies

Serum samples are analyzed for levels of total IgG, IgG subclasses, IgA, and IgM, and levels of selected specific antibodies to common vaccine antigens (Table 11).

TABLE 11
Common Vaccines Which Elicit Protective Antibody
by Age Range Initially Administered

| Birth to 6 years (bacteria) | Birth to 6 years (viruses) | Age 7 to 18 years |
|---|---|---|
| Diphtheria toxoid | Measles, Rubella | Meningococcal |
| Tetanus toxoid | Varicella Polio | polysaccharide |
| H. influenzae type B | (inactivated vaccine) | Human papilloma |
| polysaccharide | Hepatitis B, Hepatitis A | virus |
| Pneumococcal | | |
| polysaccharides | | |

The following parameters are reviewed:
Increases in levels of IgG, IgA, and IgM

For patients with sub-protective titers of specific antibody to approved microbial vaccines, the clinician and patient may decide upon revaccination, and analyze for development of protective titers post-vaccination.

### Bone Marrow Aspirates

Bone marrow aspirates are obtained from consenting patients at screening, after 4 and after 20 weeks of treatment. Aspirates are reviewed by a blinded hematopathologist and graded for cellularity and myelokathexis. If sufficient material is available, samples are analyzed for markers of neutrophil apoptosis and lymphocyte subpopulations.

The following parameters are reviewed:
Decrease in hypercellularity
Decrease in fraction of apoptotic WBC

### Clinical Assessments

Warts. Warts are monitored by photographs and/or recording of lesion location, number, and size.

Infections. Temperatures are taken twice daily, signs or symptoms of infections, resulting in fever, prompting physician visits, requiring antibiotics, or associated with hospitalization, are reviewed and compared with the year prior to treatment.

### Pharmacokinetic Assessments

If desired, pharmacokinetic assessment of blood samples for plasma levels of X4P-001 may be conducted. Blood samples are collected as scheduled. Samples are analyzed for X4P-001 concentration using reversed-phase high performance liquid chromatography (RP-HPLC) with MS/MS detection. The validated range of this bioanalytic method is 30 to 3,000 ng/ml in plasma.

Pharmacokinetics (PK) and Pharmacodynamics (PD). In order to evaluate the pharmacokinetic properties of therapy with X4P-001, levels of X4P-001, PK samples are obtained on all patients in Part A as follows
Day 1: pre-dose; post-dose at 30, 60, 90 min (each±10%) and 2, 3, 4 hr (each±15 min)
Week 5 visit: pre-dose; post-dose at 30, 60, 90 min (each±10%) and 2, 3, 4, 8 hr (each±15 min)
Week 9 and Week 13 visits: pre-dose.

Visits are scheduled for early in the day and patients are instructed to arrive at the clinic fasting and having not taken their morning dose of X4P-001.

PK are analyzed by patient and dosage regimen over the preceding week using descriptive statistics for AUC, Cmax, and Cmin.

If results suggest either (a) ongoing accumulation beyond Week 5 or (b) a specific PK parameter is associated with adverse effects, then additional sampling days may be added.

PD samples are collected on Day 1 and at Week 5 visit concurrent with scheduled PK samples (see above) for:
Total white blood cell (WBC) counts
Counts of circulating CD34+ positive cells
Assessments may include samples analyzed by flow cytometry for subpopulations of PBMCs.

If sample yields permit, additional investigational immunomodulatory subsets may be analyzed (See Table 11).

Of course, the treating physician may apply his or her professional judgment and discretion and any established standards of care, what parameters of assessment (e.g., the desired levels of ANC and ALC) should be used in determining the treatment regimen for any individual patient.

### REFERENCES

Baggiolini; 1998. Chemokines and leukocyte traffic. Nature. 392:565-568. Balabanian, et al. 2005. WHIM syndromes with different genetic anomalies are accounted for by impaired CXCR4 desensitization to CXCL12. Blood. 105:2449-2457.
Balabanian, et al. 2012. Proper desensitization of CXCR4 is required for lymphocyte development and peripheral compartmentalization in mice. Blood. 119:5722-5730.
Beaussant-Cohen, et al.; Description and outcome of a cohort of 8 patients with WHIM syndrome from the French Severe Chronic Neutropenia Registry. Orphanet J Rare Dis. 2012; 7:71.
Bohinjec, 1981. Myelokathexis: chronic neutropenia with hyperplastic bone marrow and hypersegmented neutrophils in two siblings. Blut. 42:191-196.
Broxmeyer. A WHIM satisfactorily addressed. Blood. 2014; 123:2286-8.
Cao, et al.; Effect of Low-Dose Ritonavir on the Pharmacokinetics of the CXCR4 Antagonist AMD070 in Healthy Volunteers. Antimicrob Agents Chemother. 2008; 52:1630-1634.
Crump, et al. 1997. Solution structure and basis for functional activity of stromal cell derived factor-1; dissociation of CXCR4 activation from binding and inhibition of HIV-1. EMBO J. 16:6996-7007
Dale, et al. The CXCR4 antagonist plerixafor is a potential therapy for myelokathexis, WHIM syndrome. Blood 2011; 118:4963-4966.
Dale, et al., The Severe Chronic Neutropenia International Registry: 10-Year Follow-up Report. Support Cancer Ther. 2006; 3:220-31.
Doranz 1997. Chemokine receptors as fusion cofactors for human immunodeficiency virus type 1 (HIV-1). Immunol Res. 16:15-28.
Dotta, 2011. Clinical and genetic features of warts, hypogammaglobulinemia, infections and myelokathexis (WHIM) syndrome. Current Molecular Medicine; 11:317-325. Galsky, et al. 2014. A Phase I Trial of LY2510924, a CXCR4 Peptide Antagonist, in Patients with Advanced Cancer. Clin Cancer Res. doi: 10.1158/1078-0432.CCR-13-2686.
Gulino, et al. 2004. Altered leukocyte response to CXCL12 in patients with warts hypogammaglobulinemia, infections, myelokathexis (WHIM) syndrome. Blood. 104:444-452.
Hendrix, et al. 2004. Safety, Pharmacokinetics, and Antiviral Activity of AMD3100, a Selective CXCR4 Receptor Inhibitor, in HIV-1 Infection. J Acquir Immune Defic Syndr. 37:1253-1262.
Hernandez et al., Mutations in the chemokine receptor gene CXCR4 are associated with WHIM syndrome, a combined immunodeficiency disease. Nature Genetics 2003; 34:70-74.
Kawai, et al. 2005. Enhanced function with decreased internalization of carboxy-terminus truncated CXCR4 responsible for WHIM syndrome. Exp Hematol. 33:460-468.
Kawai and Malech; 2009. WHIM syndrome: congenital immune deficiency disease. Curr Opin Hematol. 16:20-26.
Kawai, et al.; WHIM syndrome myelokathexis reproduced in the NOD/SCID mouse xenotransplant model engrafted with healthy human stem cells transduced with C-terminus-truncated CXCR4. Blood. 2007; 109:78-84. Epub 2006 Aug. 31.
Lagane, et al. 2008. CXCR4 dimerization and beta-arrestin-mediated signaling account for the enhanced chemotaxis to CXCL12 in WHIM syndrome. Blood. 112:34-44.
Lapidot and Petit; 2002. Current understanding of stem cell mobilization: the roles of chemokines, proteolytic enzymes, adhesion molecules, cytokines, and stromal cells. Exp Hematol. 30:973-981.
Ma et al. 1999. The chemokine receptor CXCR4 is required for retention of B lineage and granulocytic precursors in the bone marrow microenvironment. Immunity. 10:463-471.
Martin et al. Births in the United States, 2013. National Center for Health Statistics Data Brief. No. 175: December 2014.
McCormick, et al. 2009. Impaired recruitment of Grk6 and beta-Arrestin 2 causes delayed internalization and desensitization of a WHIM syndrome-associated CXCR4 mutant receptor. PLOS One. 4: e8102.
a-McDermott, et al.; The CXCR4 antagonist plerixafor corrects panleukopenia in patients with WHIM syndrome. Blood. 2011; 118:4957-62.
b-McDermott, et al; A phase 1 clinical trial of long-term, low-dose treatment of WHIM syndrome with the CXCR4 antagonist plerixafor. Blood. 2014; 123:2308-16.
Mosi, et al. 2012. The molecular pharmacology of AMD11070: An orally bioavailable CXCR4 HIV entry inhibitor. Biochem Pharmacology. 83:472-479.
Moyle, et al.; Proof of Activity with AMD11070, an Orally Bioavailable Inhibitor of CXCR4-Tropic HIV Type 1. Clin Infect Dis. 2009; 48:798-805.
Nyunt, et al. Pharmacokinetic Effect of AMD070, an Oral CXCR4 Antagonist, on CYP3A4 and CYP2D6 Substrates Midazolam and Dextromethorphan in Healthy Volunteers. J Acquir Immune Defic Syndr. 2008; 47:559-565.
Stone, et al.; Multiple-Dose Escalation Study of the Safety, Pharmacokinetics, and Biologic Activity of Oral AMD070, a Selective CXCR4 Receptor Inhibitor, in Human Subjects. Antimicrob Agents Chemother. 2007; 51:2351-2358.
Ward and Dale; Genetic and molecular diagnosis of severe congenital neutropenia. Curr Opin Hematol. 2009; 16:9-13.
Wong, 2008. Comparison of the potential multiple binding modes of bicyclam, monocylam, and noncyclam small molecule CXC chemokine receptor 4 inhibitors. Mol Pharmacol.74:1485-1495.
Zlotnik and Yoshie 2000. Chemokines: a new classification system and their role in immunity. Immunity. 12:121-127.
Zuelzer: “Myelokathexis”-A New Form of Chronic Granulocytopenia. Report of a case. N Engl J Med 1964, 270:699-704.

### Study Reports

A 28 day Oral (Capsule) Toxicity Study in the Male and Female Beagle Dog with a 14 day Recovery (Study CTBR77401): November 2003.
Pharmacology & Toxicology of AMD11070: Hematological Effects in the Male Beagle Dog. AnorMED, Inc. Mach 18, 2003 (Study No. AOM0031).
Hematological Effects of AMD11070 in Mice. Indiana University School of Medicine. Mar. 14, 2003 (Study No. AOM0033).
ACTG (DIAIDS) Protocol A5210. Unpublished data.

### INTERNET REFERENCES

George Diaz, Virginia Gulino. Whim syndrome. Orphanet Encyclopedia. June 2004; http://www.orpha.net/data/patho/GB/uk-Whim.pdf
NORD (National Organization for Rare Diseases) 2015: https://rarediseases.org/rare-diseases/whim-syndrome
Office of Rare Diseases: https://rarediseases.info.nih.gov/gard/9297/whim-syndrome/resources/1
Orphanet, WHIM Syndrome: http://www.orpha.net/consor/www/cgi-bin/OC_Exp.php?lng=EN&Expert=51636 Last Update October 2014
US Census Bureau: http://www.census.gov/quickfacts/table/PST045214/00 Last Revised June 2015

The invention claimed is:

1-20. (canceled)

21. A method of treating WHIM syndrome in a patient in need thereof by administering:
i) an effective amount of X4P-001 or a pharmaceutically acceptable salt or a composition thereof; and
ii) an effective amount of an agent selected from the group consisting of granulocyte colony-stimulating factor (G-CSF), granulocyte-macrophage colony-stimulating factor (GM-CSF), and intravenous immunoglobulin (IVIG).

22. The method of claim 21, wherein the X4P-001 or the pharmaceutically acceptable salt thereof or composition thereof is administered in a dose of from about 200 mg/day to about 600 mg/day in a single or divided dose.

23. The method of claim 21, wherein the agent is granulocyte colony-stimulating factor (G-CSF) or granulocyte-macrophage colony-stimulating factor (GM-CSF).

24. The method of claim 21, wherein the agent is intravenous immunoglobulin (IVIG).

25. A method of treating myelokathexis associated with a mutation in CXCR4 in a patient in need thereof by administering an effective amount of X4P-001 or a pharmaceutically acceptable salt or composition thereof.

26. The method of claim 25, wherein cells taken from the patient exhibit expression of a mutant form of CXCR4.

27. The method of claim 26, wherein cells taken from the patient exhibit increased expression of CXCR4.

28. The method of claim 26, wherein the mutant form of CXCR4 is selected from the group consisting of R334X, E343X, and S339fs342X.

29. The method of claim 28, wherein the mutant form of CXCR4 is R334X or E343X.

30. The method of claim 25, wherein the X4P-001 or a pharmaceutically acceptable salt or composition thereof is administered orally once per day.

31. The method of claim 25, wherein the X4P-001 or a pharmaceutically acceptable salt or composition thereof is administered orally twice per day.

32. The method of claim 25, wherein the amount of X4P-001 or a pharmaceutically acceptable salt or composition thereof is effective to increase absolute neutrophil count (ANC) to a level greater than or equal to 600/μL and/or to increase absolute lymphocyte count (ALC) to a level greater than or equal to 1000/μL.

33. A method of treating WHIM syndrome or myelokathexis in a patient in need thereof by administering an effective amount of X4P-001 or a pharmaceutically acceptable salt or composition thereof.

34. The method of claim 33, wherein the patient has at least one of:
i) neutropenia (ANC≤400 or ≤600/μL) and/or lymphopenia (ALC≤650 or ≤1000/μL, wherein the lymphopenia is not characteristic of other chronic neutropenias);
ii) neutropenia and chronic warts; and
iii) myelokathexis on bone marrow aspirate.

35. The method of claim 33, wherein cells taken from the patient exhibit expression of a mutant form of CXCR4.

36. The method of claim 33, wherein cells taken from the patient exhibit increased expression of CXCR4.

37. The method of claim 35, wherein the mutant form of CXCR4 is selected from the group consisting of R334X, E343X, and S339fs342X.

38. The method of claim 37, wherein the mutant form of CXCR4 is R334X or E343X.

39. The method of claim 33, wherein the X4P-001 or a pharmaceutically acceptable salt or composition thereof is administered orally once or twice per day.

40. The method of claim 33, wherein the amount of X4P-001 or a pharmaceutically acceptable salt or composition thereof is effective to increase absolute neutrophil count (ANC) to a level greater than or equal to 600/μL and/or to increase absolute lymphocyte count (ALC) to a level greater than or equal to 1000/μL.
